(19) United States
(12) Patent Application Publication
(10) Pub. No.: US 2023/0404004 A1
(43) Pub. Date: Dec. 21, 2023

(54) METHOD AND DEVICES FOR IN VITRO PLANT MATERIAL FOR GROWING AND CUTTING PLANT MATERIAL

(71) Applicant: Georgia Tech Research Corporation, Atlanta, GA (US)

(72) Inventors: Cyrus K. Aidun, Atlanta, GA (US); Else-Marie Ulrika Egertsdotter, Atlanta, GA (US)

(21) Appl. No.: 18/035,880

(22) PCT Filed: Nov. 10, 2021
(86) PCT No.: PCT/US2021/058822

§ 371 Date: May 8, 2023

Related U.S. Application Data
(60) Provisional application No. 63/111,688, filed on Nov. 10, 2020.

(51) Int. Cl. A01H 4/00

(52) U.S. Cl. CPC A01H 4/001; A01H 4/005

(57) ABSTRACT

Methods and devices for in vitro shoot growth, cutting shoots, root growth and formation of a stably growing and robust complete plants utilizing liquid culture medium and subsequently transfer to ex vitro conditions by a robotic device or other automated means are disclosed. It may comprise a temporary-immersion bioreactor system, in vitro fixtures for shoots, a holder for shoot fixtures, a cutting device and a rooting tube.

### CROSS REFERENCE TO RELATED APPLICATIONS

This application claims benefit of U.S. Provisional Application No. 63/111,688, filed Nov. 10, 2020, incorporated herein by reference in its entirety.

### BACKGROUND

Genetically improved plants in forestry, agricultural and horticultural need to be multiplied by clonal propagation to capture the genetic gains derived from breeding programs or biotechnology modifications. Clonal propagation is traditionally done by cuttings, but in vitro propagation by micro-cuttings (micropropagation) allow for scale up and automation of the methods, thereby providing cost-effective alternatives. Furthermore, large-scale propagation by cuttings (ex vitro or in vitro) is limited by biological factors in many species. For example, conifer trees are clonally propagated in vitro by somatic embryogenesis. Somatic embryogenesis is also the method of choice for in vitro propagation of many other plants. Examples include coffee and cyclamen.

In vitro cultivation of cuttings allows for manipulation of the growth environment and scale up of cutting production thus permitting plant production from many recalcitrant species. In vitro plant propagation, however, requires time consuming and repetitive manual labor associated with the risk of human error. In particular, the generation of cuttings is demanding, as it involves manual handling of individual shoots to generate cuttings that are transferred to a new culture environment for further shoot growth or shoot and root growth.

For all in vitro propagation methods, the later stages of development of the propagules are, for example (but not limited to) micro-cuttings or somatic embryos, involve root development in vitro or ex vitro, and transfer to ex vitro conditions either before or after root formation. This causes a significant bottleneck in terms of both biological development and technical handling.

Liquid culture medium has both biological and technical advantages over solidified culture medium for growth and development of plant propagules. There is a need for effective methods and devices that allow for production of a complete plant propagule by methods based on in vitro liquid culture medium that results in a fully developed propagule ready for ex vitro transfer. The plant propagule should be ready for transfer to ex vitro conditions and subsequent transfer and planting under ex vitro conditions.

### SUMMARY

Methods and devices are disclosed for in vitro shoot growth, cutting shoots, root growth and formation of a stably growing and robust complete plants utilizing liquid culture medium and subsequent transfer to ex vitro conditions by a robotic device or other automated means. It may comprise a temporary-immersion bioreactor system, in vitro fixtures for shoots, a holder for shoot fixtures, a cutting device and a rooting tube. The exemplary system and method may be used in the clonal propagation of plants.

The exemplary method and device may provide favorable environment supported by liquid culture medium for generating cuttings for in vitro shoot growth, rooting of cuttings or germination of somatic embryos in vitro or ex vitro and facilitate the subsequent transfer of such rooted or unrooted cuttings or germinated somatic embryos to the planting stage.

The method, in some embodiments, utilizes a rooting tube for support of root development.

An automated device for generating cuttings is also described. The exemplary automation systems may reduce required labor. In addition, the exemplary system may be used to provide In vitro clonal propagation that is disease free and true to type.

The rooting tube supports and protects the root part of the propagule during transfer to and at the stage of planting.

The exemplary system and method may be applied also to other propagation systems both in vitro and ex vitro for transfer of sensitive plant propagules between culture conditions.

### BRIEF DESCRIPTION OF THE DRAWINGS

The skilled person in the art will understand that the drawings, described below, are for illustration purposes only.

FIG. 1A illustrates the devices of tubes for positioning the plant cuttings held by the holder-mesh at the start of the TIB process involving rooting and shoot multiplication and ending with transfer to ex vitro conditions. The process at the start of the method applied for use with the devices is outlined herein.

FIG. 1B shows an example method of using a precision non-contact cutting system to cut a tube housing and a grown propagule and root in accordance with an illustrative embodiment.

FIG. 1C shows a plant growing system that can be employed in the temporary immersion bioreactor growth process.

FIG. 2 illustrates a system-level schematic of a non-contact cutting system in accordance with an illustrative embodiment. The system of FIG. 2 can be used to cut in vitro propagules without contact, and therefore, to avoid any contamination. This system may also be used to cut the roots of the germinant developed from somatic embryos, since in some cases it is desired to develop the root from the cuttings and not the somatic embryos.

FIGS. 3A-3G each shows different aspects of the non-contact cutting system in accordance with an illustrative embodiment.

FIGS. 4A-C show a V(vertical)-germination bioreactor in several views. This research model holds 30 tubes for germination of somatic embryos, shoot multiplication or rooting of cuttings.

FIG. 5 is an image of a V-germination bioreactor showing root growth of germinated Norway spruce somatic embryos inside planting-tubes and shoot development.

FIG. 6 shows the germination plant box. The V-germination bioreactor shell is shown without connections with test propagules (Araucaria cuttings) for use with the automated Germination Platform. This plant box can hold 1080 tubes for germination of somatic embryos, shoot development or rooting of cuttings. FIG. 6 shows the entire box and a blown-up image showing the individual tubes with plants in them.

### DETAILED SPECIFICATION

### Definitions

By “artificial plant seed” is meant a plant seed which does not occur in nature but rather is a plant propagule functionally similar to a plant seed that has been produced by some level of human intervention using micropropagation techniques. The “artificial plant seed” is able to regenerate into a plant and may undergo germination. The terms “artificial plant seed” and “artificial seed” may be used interchangeably herein and may refer to but not limited to somatic embryos.

By “rooting tube” is meant any vesicle meant to contain a plant propagule. The terms tube refers to a tubular structure that can be of various materials, such as cellulose, paper, plastic or biodegradable material depending on what is required for the specific application, plant species and propagation system. The rooting tube is specifically designed, in some embodiments, in terms of its physical and chemical parameters to accommodate the rooting of the propagule.

By “micropropagation” is meant propagation of plants by growing plantlets in tissue culture and then planting them out.

By “temporary immersion bioreactor” or “TIB” is meant systems in which the entire culture or plant tissue is wetted with nutrient solution and then the excess nutrient is drained so that proper aeration is provided to the cultured tissue.

For purposes herein, the term ‘propagule’ is defined as a plant shoot cutting either in vitro or ex vitro, any other plant derived part that can be used to generate a plant, or a somatic embryo of any plant species.

“Cutting” refers to a detached plant part that under appropriate cultural conditions can regenerate the complete plant without a sexual process.

“Ex vitro” refers to organisms removed from tissue culture and transplanted: generally plants to soil or potting mixture.

“Inoculum” refers to a small piece of tissue cut from callus, or an explant from a tissue transferred into fresh medium for the continued growth of the culture.

“In vitro” refers to living in test tubes, outside the organism or in an artificial environment, typically in glass vessels in which cultured cells, tissues, or whole plants may reside.

“In vivo” means the natural conditions in which organisms reside. Refers to biological processes that take place within a living organism or cell under normal conditions.

“Medium” refers to the liquid or solidified formulation upon which plant cells, tissues or organs develop.

“Medium Formulation” refers to, in tissue culture, the particular formula for the culture medium. It commonly contains macro-elements and micro-elements, some vitamins (B vitamins, inositol), plant growth regulators (auxin, cytokinin, and sometimes gibberellin), a carbohydrate source (usually sucrose or glucose), and often other substances, such as amino acids or complex growth factors. Media may be liquid or solidified with agar; the pH is adjusted (ca. 5-6) and the solution is sterilized (usually by filtration or autoclaving). Some formulations are very specific in the kind of explant or plant species that can be maintained; some are very general.

“Meristem” refers to undifferentiated tissue, the cells of which are capable of active cell division and differentiation into specialized and permanent tissue such as shoots and roots.

“Micronutrient” refers to an essential element normally required in concentrations<0.5 millimole/liter.

“Radicle” refers to that portion of the plant embryo which develops into the primary or seed root.

“Scarification” refers to the chemical or physical treatment given to some seeds (where the seed coats are very hard or contain germination inhibitors) in order to break or weaken the seed coat sufficiently to permit germination.

“Sterile” refers to the medium or object with no perceptible or viable micro-organisms.

“Sterilize” is the process of elimination of micro-organisms, such as by chemicals, heat, irradiation or filtration.

“Terminal bud” is located at the tip of a stem (apical is equivalent but rather reserved for the one at the top of the plant)

“Transverse” is across the width of the explant, the direction perpendicular to the ridge, the perpendicular side of the longitudinal side.

It must also be noted that, as used in the specification and the appended claims, the singular forms “a,” “an” and “the” include plural referents unless the context clearly dictates otherwise. Ranges may be expressed herein as from “about” or“5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 44.24, 4.24-5, 2-5, 3-5, 1-4, and 2-4). It is also to be understood that all numbers and fractions thereof are presumed to be modified by the term “about.”

Example System for Generating and Supporting Shoot Cuttings and Plant Propagules

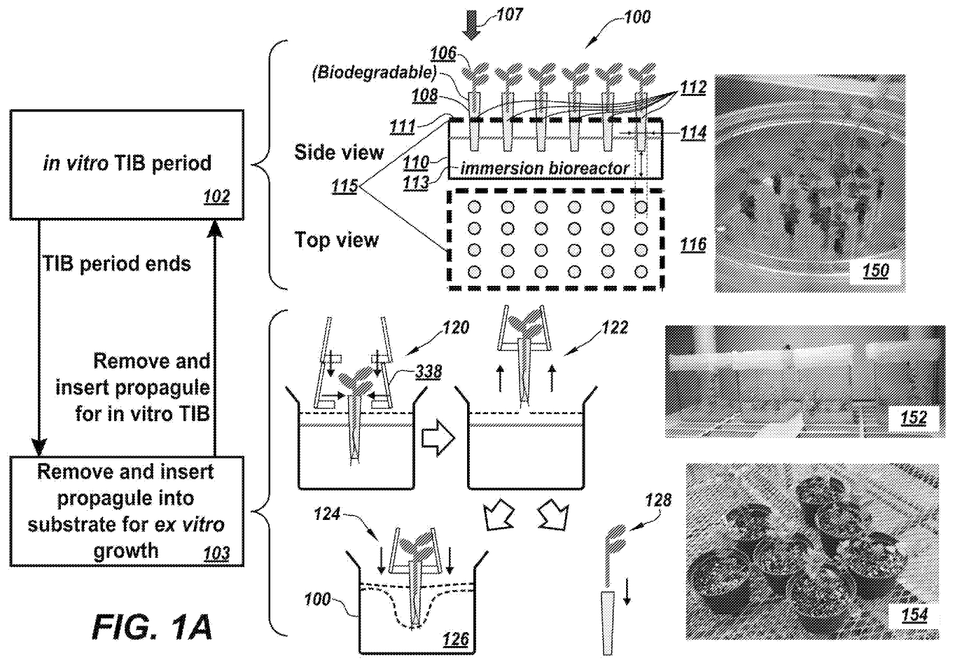

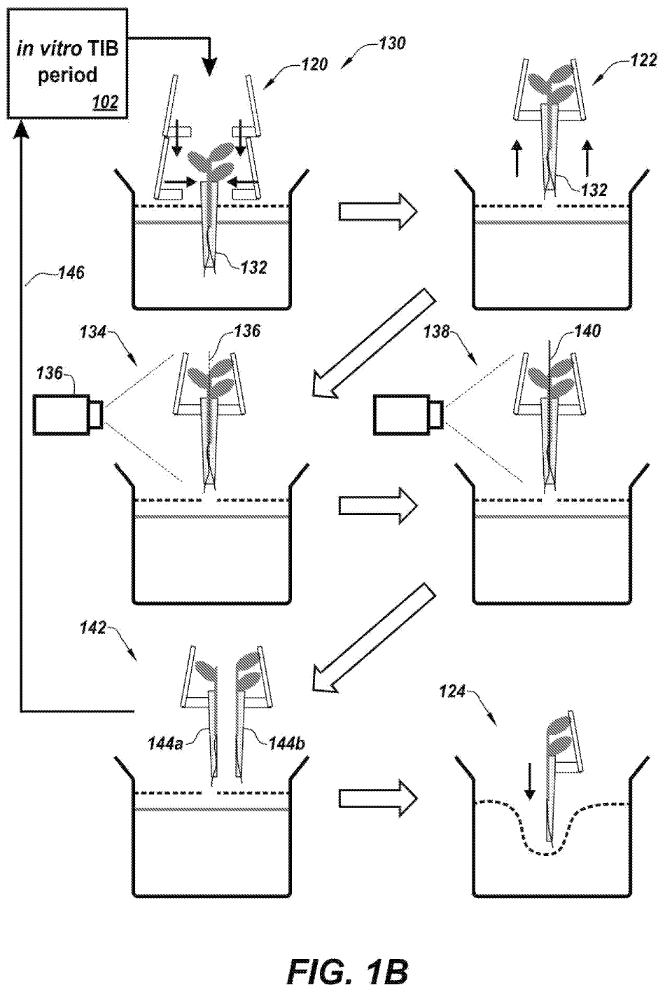

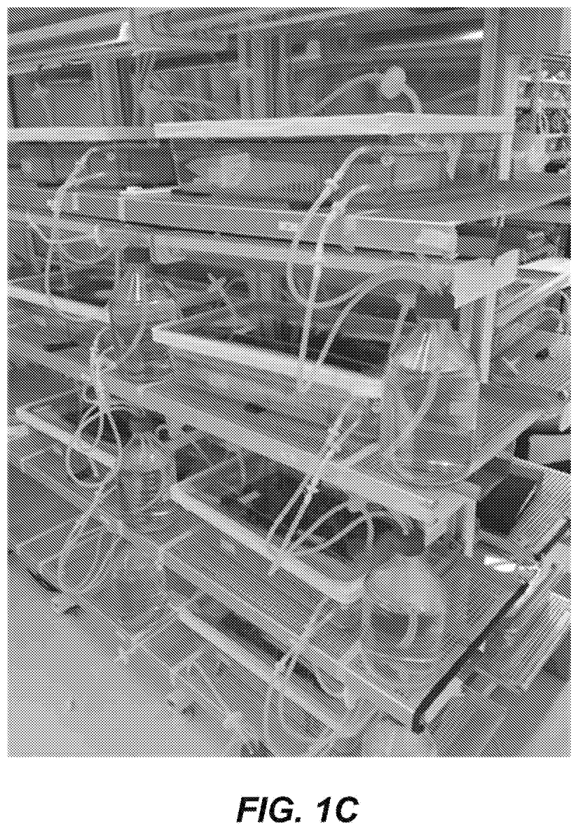

FIG. 1A shows an example construction and use of an immersion bioreactor device 100 for propagating plants in accordance with an illustrative embodiment. The immersion bioreactor device 100 can be utilized with a method including processes that cover the in vitro TIB period until the propagule is inserted into the substrate for ex vitro growth or for new shoot generation (shown as process 102, 103, and 104).

In the example shown in FIG. 1A, Process “1” 102 involves positioning (107) a propagule 106 into a rooting tube 108 (e.g., biodegradable rooting tube 108) (hereafter also referred to as “tubes” 108) at a desired level inside the tube. The tubes are then affixed into a tube-holder 110 where the top of the tube-holder 110, e.g., a mesh or a plate 111, has formed cavities or openings 112 that are dimensioned to fit the tubes 108 at a diameter 114 of the tube 114 to position the tube 108 at an optimal vertical position 116 relative to the holder 110. The holder 110 include a temporary immersion bioreactor 113 to facilitate growth of the roots. The tubes 108 are arranged (115) in a pattern that is compatible with the geometrical design of the tray receiving the rooted propagules ex vitro (116). The holder 110 with tubes 108 and temporary immersion bioreactor (TIB) 113 are transferred to a storage area with a pre-defined light source for rooting and shoot development and/or for mature somatic embryo germination.

It has been observed that a substantial number of shoot cuttings can be lost during the excision of the shoot cuttings from its rooting tube. The rooting tube 108 can be made of a biodegradable material so the shoot cutting can be retained in the tube when being planted without the shooting being excised from the tube 108, thereby improving the yield of the processing.

In addition, the device 100 comprising the tube 108 and with the grown propagule 106 and root can be cut using a precision non-contact cutting system to increase the yield of the growth. Referring to FIG. 1A, “Process 2” 103 may commence when the process of forming a viable root and a shoot is complete, and the target for the process is the planting of the propagule. The tube holder 110 is removed (shown with operations 120, 122) from the TIB and the holder 110, and the tubes 108 holding the rooted propagules can be exactly positioned (124) into a substrate 126 in a new container 125. In some embodiments, the rooted propagules and holder can be moved to a transport holder.

As an alternative to, or in combination with “Process 2” 103, “Process 3” 104 may commence when the process of forming enough shoot for shoot cuttings is complete and the target for the process is generating cuttings. The propagule inside the holder has formed a sufficiently developed shoot that such can be cut to generate new shoots. In some embodiments, the grown propagule inside the holder can be cut using a non-contact cutter, e.g., laser or water cutter.

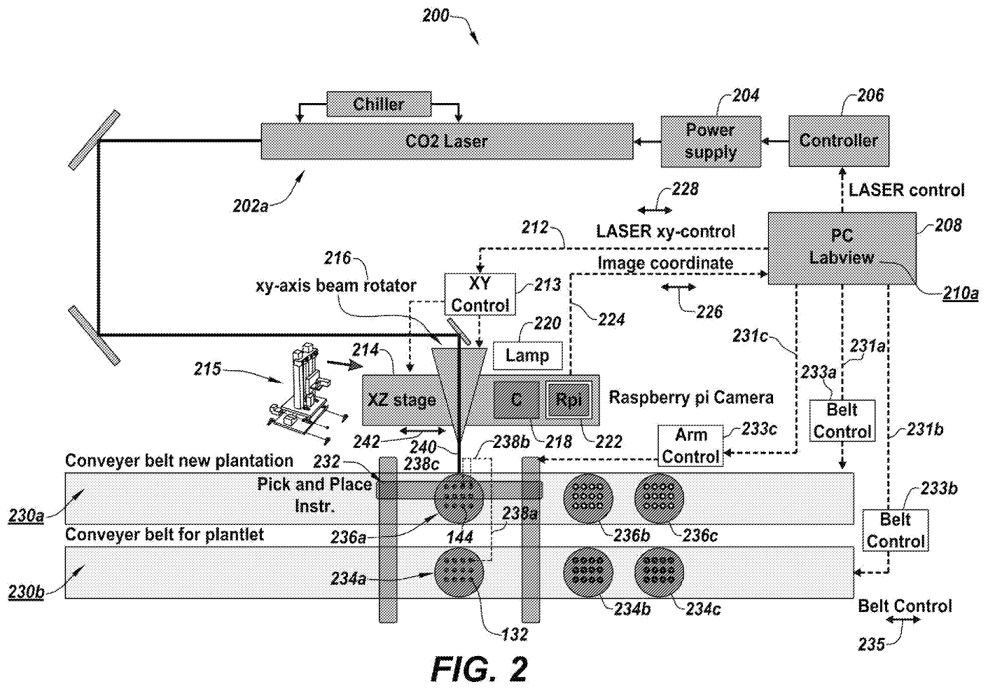

FIG. 1B shows an example method 130 of using a precision non-contact cutting system 200 (see FIG. 2) to cut a tube 108 housing and a grown propagule 106 and root (hereinafter referred to as a tubed propagule 132) in accordance with an illustrative embodiment. In the example shown in FIG. 1B, the tubed propagule 132 is removed (shown with operations 120, 122) from the TIB and the holder 110. The precision non-contact cutting system 200 comprises an aligning system 202, e.g., comprising one or more cameras (136) and controllers, to position (134) the propagule 106 with respect to a non-contact cutting element (not shown—see FIG. 2). The precision non-contact cutting system 200 may acquire images of the tubed propagule 132 to identify a main axis component (138) for the propagule 106 to direct the cutting (138) of the tubed propagule 132 along that axis 138. Subsequent to the cutting, the bisected tubed propagules 144 (shown as 144a and 144b) can be placed into a holder to be either positioned (124) into a substrate for growth or to be provided (146) as input to the TIB processing (102) to generate additional generations of a propagule with a viable root and a shoot.

Indeed, the systems, methods, and devices described for generating as well as further supporting shoot cuttings or other types of plant propagules like mature somatic embryos during the rooting process. These processes can equally support the transfer of unrooted shoot cuttings produced utilizing liquid culture medium for transfer to ex vitro for rooting. A key part of some embodiments is the rooting tube for support of the rooting process and support and protection during transfer to the planted stage. Importantly, this rooting tube can be made of biodegradable materials. The cutting may be generated by a specially designed device described below. First, the cutting or other plant propagule is positioned into the tube fixed inside the holder. The propagule inside the tube is treated with liquid medium of a composition suitable for the species and type of propagule to induce rooting by the operation of the temporary immersion bioreactor (TIB).

The tube is designed, in some embodiments, of a material that support stimulation of root formation through contact with the liquid medium. The tube material can be biocompatible and biodegradable.

The tube is designed, in some embodiments, to hold the propagule inside the tube with easy access for positioning of the propagule from the top. The tubes may be transferred by any suitable mechanism or by a holder that takes the number of tubes suitable for the temporary-immersion system in use. The holder can be comprised of mesh, wherein some grid-spaces in the mesh hold the tubes directly, and others are holding the tube-grids to form a continuous mesh. The holder-mesh is contained, in some embodiments, within a supporting tray that allows for positioning of the propagules into the tubes outside the TIB, wherein the tray can be lifted with the tubes and propagules inserted for transfer to and from the TIB. The tube and holder design provides an effective means of inserting the plant propagules into and out of the TIB that can also be automated for large-scale operations. The specific design of the tube allows for optimized rooting of plant propagules and novel rooting from recalcitrant species.

In cases where the cutting of the shoot is required for in vitro propagation, the embodiments may comprise a non-contact cutting mechanism. This mechanism is based, in some embodiments, on a laser beam that is formed such that cuts the shoot at a precise location from a distance without contact. The laser beam is focused, in some embodiments, in a way that the cutting is fast and precise. Devices and mechanisms used in the cutting process allows holding the plant in position by gripping, air suction, or other method that keeps the plant in position for the plant to be cut, imaging the plant and after image analysis and in silico image analysis and positioning processes, cutting at a precise location.

Devices and methods for generating shoot cuttings, growing and rooting shoot cuttings, or rooting somatic embryos, transfer of plant propagules produced utilizing liquid culture medium being shoot cuttings, other propagules or somatic embryos to a TIB system for rooting and subsequent transfer of the same plant materials at a more developed stage from the TIB to ex vitro conditions, or a transfer within the ex vitro condition, and planting in substrate without directly handling the plant propagules in the intermittent period from deposition, during TLB culture and until planted, are described herein.

The exemplary system and method may be used independently with generic temporary-immersion bioreactor (TIB) systems or, e.g., specifically with the Bioautomaton Systems Inc (BSI) temporary-immersion bioreactors (Mamun et al. 2018; Businge et al. 2017) or Germination Platform as described in patent WO2016/098083 (PCT/IB2015/059811) or BSI's V-Germination bioreactor.

### Example Non-Contact Cutting System

FIG. 2 shows an example precision non-contact cutting system 200 comprising a non-contact cutting and mechanized handling system in accordance with an illustrative embodiment.

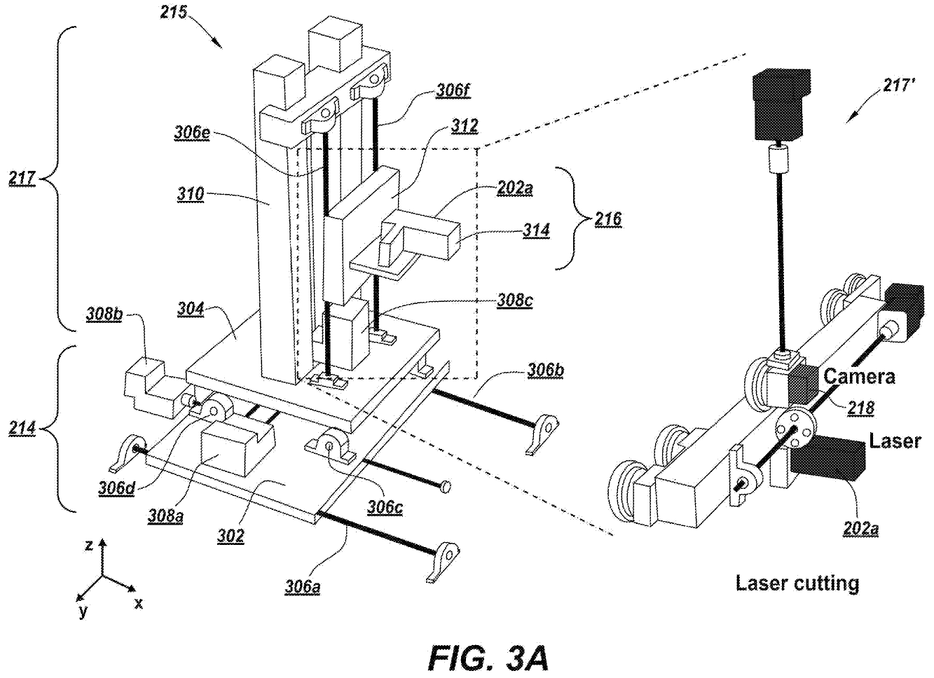

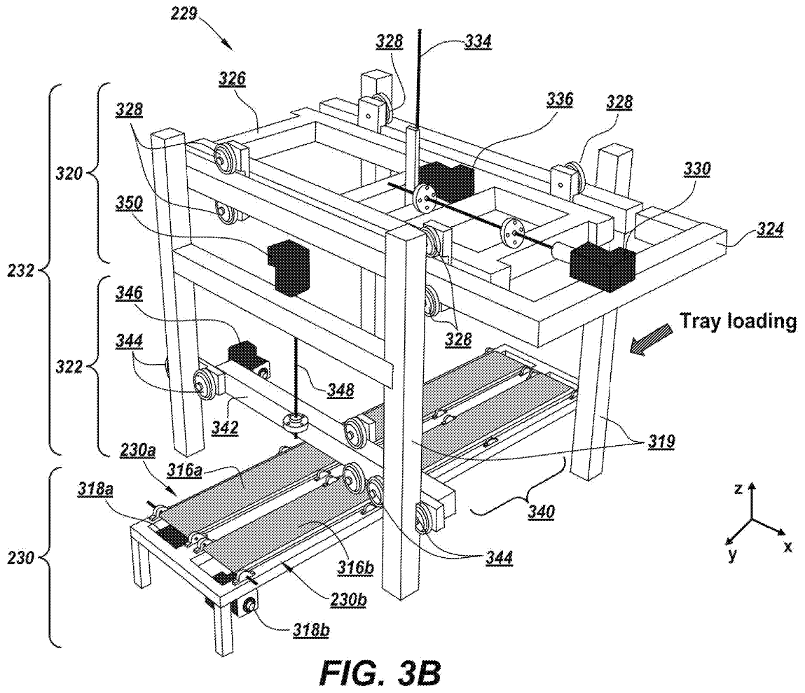

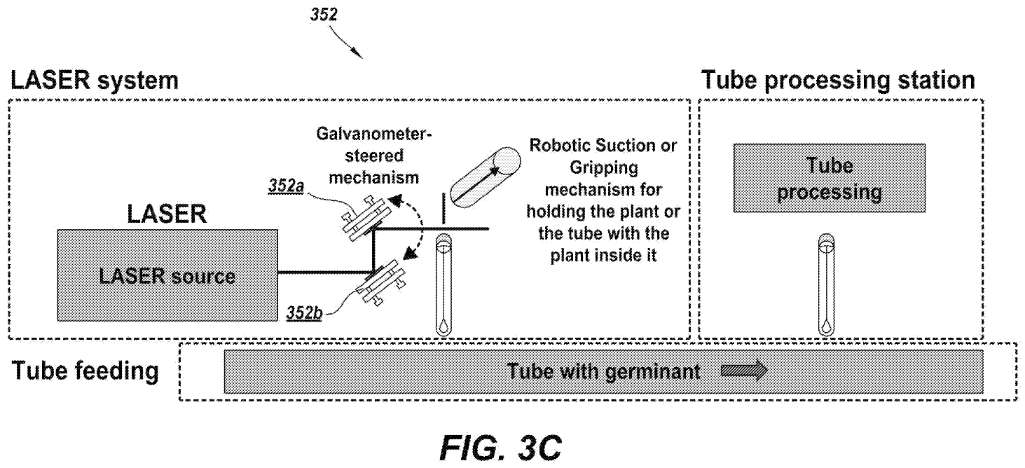

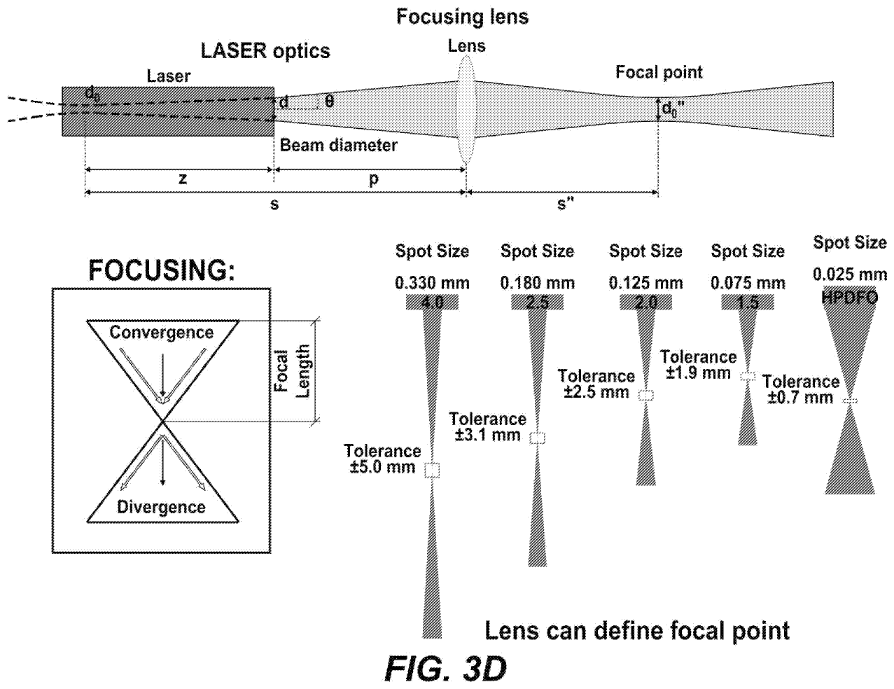

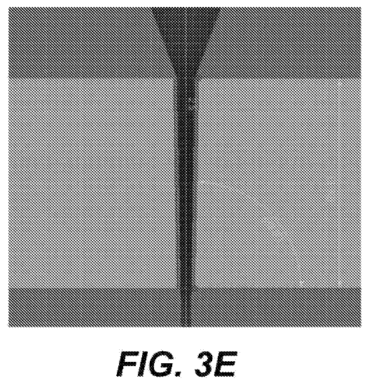

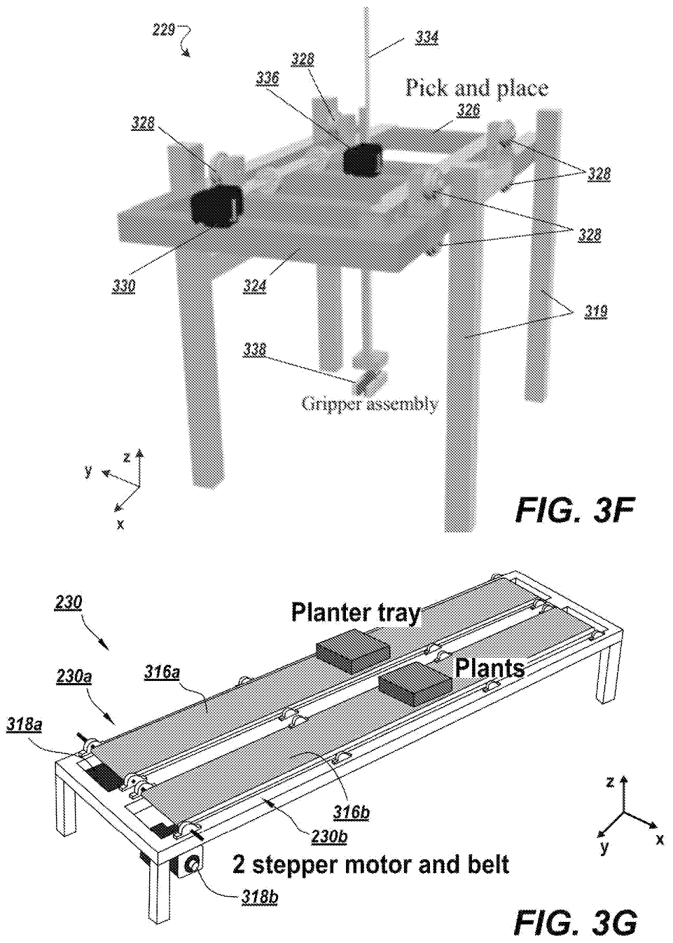

The non-contact cutting system 200 comprises a non-contact cutting unit 202 (shown as “CO2 Laser” 202a) that is coupled to a power supply 204 and controller (206). The controller 206 is connected to a central computing device 208 (shown as a “PC” 208) executing an automation program 210 (shown executing in a program environment, “Labview” 210a). The central computing device 208 includes one or more electronic interface boards (not shown) that operatively connects (212) to XY laser assembly 215 comprising a XZ assembly controller (shown as “XY Control” 213) that directs the control of linear actuators of a laser XY stage 214 (shown as “XY Stage” 214) and a second laser translation stage 217 (shown as “xy-axis beam rotator 216). The XY laser assembly 215 further includes an image acquisition system comprising a camera 218 (shown as “C” 218, lamp 220, and camera controller 222 (shown as “Rasberry pi Camera” controller 222). FIG. 3A, discussed below, provides a detailed view of the XY laser assembly 215. The camera controller 222 is connected (224) to the central computing device 208 and is configured to process the images from the camera 218 to identify, e.g., main axis component/cutting axis 136 of, a propagule 106 (see FIGS. 1A, 1B) and its root in a tubed propagule 132 (see FIGS. 1A, 1B). In the example of FIG. 2, the camera controller 222 is configured to provide image coordinates 226, e.g., of cutting axis 136, to the automation program 210a. The automation program 210a can receive the image coordinates 224 and use it to send xy-control signals or command 228 (shown as “LASER xy-control” 228) to the automation program 210a. In other embodiments, the controller 222 (or the camera 218 may directly) provide images to the automation program 210a to determine the cutting axis.

The non-contact cutting system 200 includes a processing stage 229 comprising one or more tray holder transition system 230 (shown as “Conveyor belt new plantation” 230a and “Conveyor belt for plantlet” 230b) and a pick-and-place instrument 232. In the example shown in FIG. 2, the central computing device 208 includes interface boards (not shown) that operatively connects (231a, 231b, 231c) to one or more controllers 233 (shown as “Belt Control” 233a, 233b and “Arm Control 233c”) of the holder transition system 230 and pick-and-place instrument 232. FIG. 3B, discussed below, provided a detailed-view of the processing stage 229 comprising the tray holder transition system 230 and the pick-and-place instrument 232. FIGS. 3F and 3H shows separate views of the processing stage 229 and the tray holder transition system 230.

The tray holder transition system (e.g., 230b) is configured to translate and position one or more trays 234 (shown as 234a, 234b, 234c) having the grown tubed propagule 132, e.g., based on belt control signals 235 received from the automation program 210a. In the example shown in FIG. 2, the tray holder transition system 230 includes a second conveyor belt 230a to hold a second tray 236 (shown as 236a, 236b, 236c) that receives the bisected tubed propagules 144 from the first tray 234 once cut. The pick-and-place instrument 232 is configured to pick up (238a) and hold (238b) a plantlet (e.g., 132) to be cut by the laser beam 240 from the laser source 202a, and then place (238c) the plantlets 144 in the tray 236a receiving the cut propagules or the cut plants. To improve the speed of the processing, the automation program 210a can direct the laser XY stage 214 to position (242) the beam output (shown via beam 240) in coordination with the pick-and-place instrument 232.

### Example XY Laser Assembly of the Non-Contact Cutting System

FIG. 3A provides a detailed view of the XY laser assembly 215. In the example shown in FIG. 3A, the XY laser assembly 215 includes the laser XY stage 214 and the second laser translation stage 217. The laser XY stage 214 comprises a first stage 302 and a second stage 304 that are mounted on displacement shafts 306 (shown as 306a-306d) and mechanically coupled to linear actuators 308 (shown as 308a, 308b) (e.g., controlled by 213) to provide displacement of the laser source in the x and y direction. The second stage 304 provides a base to and is coupled to a gantry 310 that houses the second laser translation stage 217 and a non-contact cutting source 202 (shown as 202a). The second laser translation stage 217 includes a third stage 312 that is mounted on displacement shafts 306 (shown as 306e, 306f) and mechanically coupled to linear actuators 308 (shown as 308c) (e.g., controlled by 213) to provide displacement of the laser source in the z-direction. The output of the non-contact cutting source 202a is shown as 314. Another embodiment of the second laser translation stage 217 (shown as 217′) is also shown in FIG. 3A. The second laser translation stage 217′ is configured to move the non-contact cutting source 202 (shown as “laser” 202a′) in a z-direction by moving the beam output along that axis.

### Example Processing Stage of the Non-Contact Cutting System

FIG. 3B provided a detailed-view of the processing stage 229 comprising the tray holder transition system 230 (shown as 230a, 230b) and the pick-and-place instrument 232. The tray holder transition system 230a, 230b includes respective belts 316a, 316b that are driven by motors 318a, 318b. In the example shown in FIG. 3B, the pick-and-place instrument 232 includes multiple pick-and-place stages, mounted to a single gantry 319, including a first pick-and-place stage 320 and a second pick-and-place stage 322. The first pick-and-place stage 320 is configured to pick up (e.g., 238a) and hold (e.g., 238b) a plantlet (e.g., 132) to be cut by the laser beam 240 from the non-contact cutting source 202, and then place (e.g., 238c) the plantlets 144 in the tray 236a receiving the cut propagules or the cut plants. The second pick-and-place stage 322 provides secondary sorting or moving operations, if needed.

The first pick-and-place stage 320 is mounted at the top of the gantry 319 at gantry beams 324. The gantry beams 324 support a runway rail assembly 326 that is configured to move in the x-direction. The runway rail assembly 326 seats on the gantry beams 324 on wheels 328 and are actuated by motor 330 (e.g., controlled by 233c). The runway rail assembly 326 includes a trolley 332 (not shown) that is mounted on a shaft 334 that is actuatable by motor 336 (e.g., controlled by 233c).

During operation, the automation program 210a can direct the conveyor belt 316a, 316b to move trays 234, 236 (in the y-direction) into the processing area 340. The automation program 210a then directs controller 233c to (i) lower the trolley 332 comprising a mechanical arm (e.g., grip) and/or suction unit 338 (see FIGS. 1A, 1B, 3F) (along the shaft 334 in the z-direction) to a position above a plantlet 132 and (ii) pick up its tube 132 with the mechanical arm and/or suction unit 338. The automation program 210a then directs controller 233c to raise (in the z-direction) and translate (in the x-direction) the trolley 332 to a position for cutting by the non-contact cutting source 202. The automation program 210a then directs the cutter controller 206 to output a beam (e.g., laser) or stream (e.g., water) to cut the tubed propagule 132 into the bisected tubed propagules 144. FIG. 3C, discussed below, shows an example configuration/assembly of the non-contact system to provide a planar cutting 140 along the axis 136. The automation program 210a then directs controller 233c to (i) position (by moving in the x-direction) the bisected tubed propagules 144 over an available slot in tray 236, (ii) lower the bisected tubed propagules 144 to the slot, and (iii) release the bisected tubed propagules 144. In some embodiments, the automation program 210a can direct the bisected tubed propagules 144 to be placed in separate slots of the try 236.

Referring still to FIG. 3B, the second pick-and-place stage 322 can be employed to provide secondary sorting or moving operations. In the example shown in FIG. 3B, the second pick-and-place stage 322 is mounted at an exit side of the gantry 319. The second pick-and-place stage 322 has a second runway rail assembly 342 that is (i) movable coupled rails of the gantry 319 via wheels 344 and (ii) actuated by motor 346 (e.g., controlled by 233c). The second runway rail assembly 342 includes a second trolley 344 (not shown) that is mounted on a shaft 348 actuatable by motor 350 (e.g., controlled by 233c).

Indeed, the processing stage 229 can be used to process batches of trays. The processing stage 229 can be fed and empty by an operator or by other conveying systems. Other configuration of the processing stage 229 may be employed, including, e.g., different configuration of actuations, having additional stages for processing, etc.

### Axial Laser Cutter Configuration

FIG. 3C shows an example assembly/configuration of the non-contact beam cutter, e.g., xy-axis beam rotator 216, to provide planar cutting 140 along the axis 136 using a beam/stream output. In the example of FIG. 3C, the xy-axis beam rotator 216 includes one or more steering mirrors 352 (shown as 352a, 352b). The XZ assembly controller 213 can direct the steering mirrors 352 to rotate and adjust the output direction of the beam along the cutting plane of 140. In some embodiments, the steering mirrors 352 employs galvanometer-based steering-based controls.

In some embodiments, the non-contact cutting system 200 may be placed in an enclosure to provide a sterile or controlled environment for the cutting.

In some embodiments, the non-contact beam cutter may employ a CO2 type LASER. Examples of CO2 type laser includes DC Glass Laser Tube and RF Metal Laser Tube. Other types of laser sources may be used. Example outputs of the laser can be 40 W, 60 W, 80 W, 100 W, among others. Other power output can be used with appropriate configurations (e.g., longer cut time).

FIG. 3D shows example configuration for laser optics. In one example, the non-contact beam cutter may employ a 100 W RF laser to generate a circular beam having a size of 2 mm diameter and a power density at the focal point of about 3.18e+3 W/cm². Other spot sizes may be used, e.g., as shown in FIG. 3D, among others.

FIG. 3E shows example cut speed of the non-contact beam cutter. The energy level J at a given power can be defined as the power P of the laser multiply by the laser focal surface d divided by the translation speed V per Equation 1.

$$J = \frac{P * d}{V} \qquad ( Equation 1 )$$

For example, for a laser beam to pierce a 3 mm-thick material, FIG. 3E shows an exaggerated Kerf angle of 1 deg and a kerf width of 0.25 mm. With a Pₘₐₓ of 80 W, a focal distance d of 250 μm, the maximum translation speed is 33 cm/s. Appropriate configuration of the cutting speed can be employed using Equation 1. An example CO₂ laser that may be used is compact CO₂ laser system manufactured by Synrad or the CX-series CO₂ laser manufactured by Coherent.

Other types of commercially available gas laser (in addition to CO₂ lasers, e.g., Helium (He), Neon (Ne), argon ion, carbon monoxide (CO), excimer lasers, nitrogen (N) lasers, hydrogen (H)) as well as solid state lasers (e.g., cerium (Ce), erbium (Eu), terbium (Tb), sapphire (Al₂O₃), neodymium-doped yttrium aluminum garnet (Nd:YAG), Neodymium-doped glass (Nd:glass) and ytterbium-doped glass, neodymium-doped yttrium aluminum garnet (Nd:YAG)) may be employed. Similarly, semiconductor of liquid type lasers may be employed.

And as noted throughout the specification, other non-contact cutting mechanisms may be used, e.g., water cutting, e.g., using a water jet cutter. In some embodiments, contact-based cutting may be employed.

### Preparation of Plant Tissue Fragments

The present invention is based on devices, methods and systems for preparation of plant tissue fragments that are able to regenerate into a plant or plant tissue that overcomes the obstacles of high production cost and non-sterile environments. Specifically, the techniques described herein allow for the automated handling of plant material so that the need for human touching and handling is minimized. This drastically reduces cost and allows the plant to be kept in a sterile environment. In other broad aspects, disclosed herein is an automated system that is capable of both cutting and propagating plant material. In particular embodiments, the methods, devices, and systems of the present invention produce plant tissue that is able to regenerate, allowing for the rapid multiplication of plants. A particular advantage provided by the fragments of the invention is successful production of plants in high frequency directly from small fragments.

Plant tissue culture has been used extensively in plant propagation, transformation, mutagenesis, breeding and virus elimination. Such tissue culture systems are generally referred to as “micropropagation” systems, wherein plant tissue explants are cultured in vitro in a suitable solid or liquid medium, from which mature plants are regenerated. In particular embodiments, “micropropagation” relates to conventional micropropagation technology or alternatively, artificial plant seed technology.

The present invention is applicable to a number of different plant tissues inclusive of leaf spindle or whorl, leaf blade, axillary buds, stems, shoot apex, leaf sheath, internode, petioles, flower stalks, embryo, root or inflorescence. Suitably, a relevant biological property of the plant tissue used in the present invention is that they contain actively dividing cells having growth and differentiation potential. Preferably, the plant tissue is axillary bud and/or shoot apex. In preferred embodiments, the shoot apex is apical bud tissue and/or apical meristem tissue.

### Example TIB Devices

The overall construction of the device is presented in FIGS. 1A and 1B. Generally, FIGS. 1A and 1B illustrate the devices of tubes for positioning the plant cuttings from a plant held by the holder-mesh at the start of the temporary immersion bioreactor (TIB) process involving rooting and shoot multiplication and ending with transfer to ex vitro conditions. The same process can be applied for mature somatic embryos to germinate and form a root and a shoot.

Exemplary automated and non-automated bioreactors are described, for example, in U.S. Publication Appl. No. US20040209346 which shows an intermittent immersion bioreactor consisting of a central pivoted mechanism whose operation is automated. PCT Application WO2012061950 details an automated bioreactor to obtain a kind of Antarctic species which requires special and well controlled conditions for micropropagation. PCT Application WO2012044239 describes a bioreactor consisting of a container comprising an upper compartment (for plant tissue to be propagated), and a lower compartment (for liquid nutrient medium) with the liquid being transported through a gas injection compartment, from the lower compartment to the upper compartment, in accordance with the programming of the immersion period. PCT Application WO2012156440 describes a temporary immersion bioreactor system, in which each bioreactor is composed of two containers, the upper being intended for the material to be propagated, and the lower for the liquid nutrient medium which is transported to the upper container for the completion of the soaking cycle, with the latter system characterized by maximum utilization of space of the micropropagation environment. These references are incorporated herein in their entirety for their teachings concerning TIBs.

Specifically, the device disclosed herein comprises a TIB, wherein said bioreactor comprises: a tube holder 110, wherein said tube holder comprises a bottom layer as a supporting tray, and a top layer comprising a tube receiver 111, wherein said tube receiver comprises cavities 112 sized and configured for receiving tubes 108; and two or more tubes, wherein said tubes are formed of biodegradable material, and further wherein the tubes are removably engaged by the cavities of the tube receiver, wherein a proximal end of the tube is open for receiving a plant propagule, and the distal end of the tube resides in the supporting tray of the tube holder.

This device, particularly the tube holding the plant propagule, can be made of material that supports stimulation of root formation. The TIB can generally comprise an upper container for receiving tubes containing the propagated material and a lower container (supporting tray) for the nutrient medium destined for immersion of the material in vegetative propagation. The containers have a suitable format to maximize space utilization in the environment for the micropropagation, for example, in a shape of a substantially rectangular or cubic box, preferably a rectangular box. The walls of the containers can be opaque or transparent and can be made of suitable material, not only to allow the passage of light, but also to respond positively to tests of biological functionality and structural and thermal resistance, for example, acrylic material of polyethylene, polypropylene, polycarbonate and glass, preferably with high mechanical strength. Any components of the device can be made from Teflon, aluminum, safety glass, or other material known to those of skill in the art. Furthermore, components of the device can include sensing and feedback control components. For example, the tray moving mechanism can comprise a feedback sensor such that when the tray has reached a desired place, the sensor can send feedback to the motor to stop moving. There can also be an emergency stop mechanism as part of the device.

The propagule can be manipulatable inside the tube via the proximal end. In other words, a plant propagule, such as an artificial seed, can be placed inside the tube by either machine process or the hand of man. A detailed description of the machine process is described below. The tube can be permeable, or semi-permeable, such that liquid media in the supporting tray in which it is placed may come into contact with all or a portion of the plant propagule. For example, the tube may be made of porous material, or may have 1, 2, 3, 4, 5, 6, or more openings in the tube which allow for the exchange of material into and out of the tube. Alternatively, the tube may be comprised of a solid material such that the growth media is wholly contained within the tube.

As a particular advantage, the rooting tube (as seen in FIG. 15, also referred to herein as the “tube”) can be made of biodegradable material. Examples of such material include, but aren't limited to, naturally occurring material such as wood cellulose fiber or compressed compost, or biologically synthesized plastics (also called bioplastics or biobased plastics) and petroleum-based plastics. Examples of biologically synthesized plastics, which are plastics produced from natural origins, such as plants, animals, or micro-organisms, include polyhydroxyalkanoates (PHAs); polylactic acid (PLA); starch blends; cellulose-based plastics; and lignin-based polymer composites. Petroleum-based plastics are derived from petrochemicals, which are obtained from fossil crude oil, coal or natural gas. The following petroleum-based plastics are biodegradable: polyglycolic acid (PGA); polybutylene succinate (PBS); polycaprolactone (PCL); poly(vinyl alcohol) (PVA, PVOH); and polybutylene adipate terephthalate (PBAT). The biodegradable material, in some embodiments, is configured to retain its shape for the TIB period.

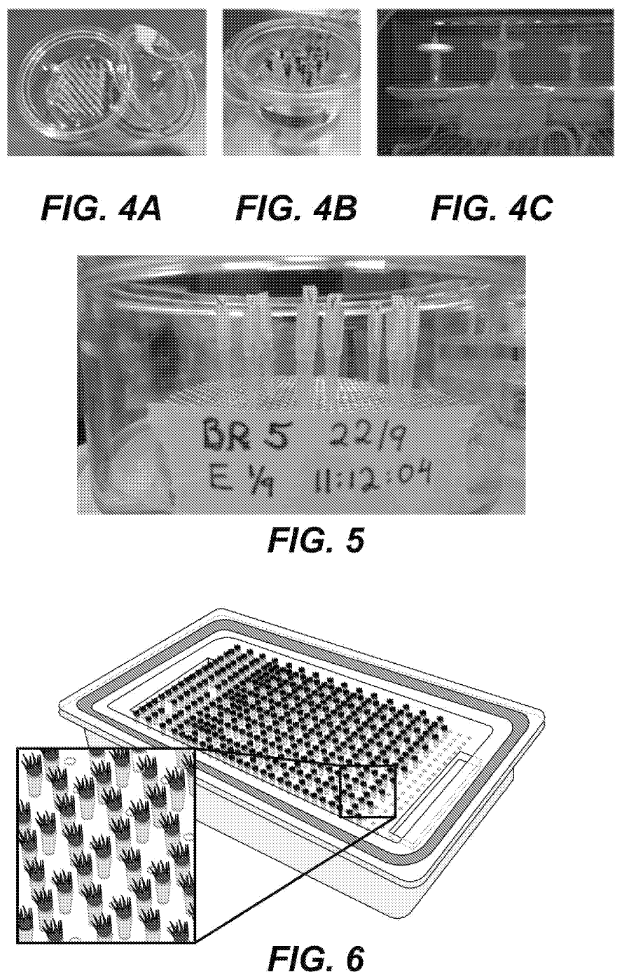

The tube receiver disclosed herein (shown in FIGS. 1A, 1B, 4A-C, 5, and 6A-B) is designed to receive and hold the tubes disclosed herein in place. It can be made from a variety of materials, and can be solid or made of a mesh so that it has some flexibility to it. The tube receiver has cavities, or holes, throughout, which are sized and shaped for receiving a tube. The tube receiver can have any number of holes in it, such as 2, 10, 20, 30, 40, 50, 60, 70, 80, 90, 100, 200, 300, 400, 500, 600, 700, 800, 900, 1000, 1500, 2000, 2500, 3000, or any amount below, above, or between these amounts. In one specific embodiment, the tube receiver has 1,080 cavities for receiving tubes. These can be spaced at any interval, and at any pattern, known to those of skill in the art. For example, they can be equidistant, in rows, or any other format. Importantly, the cavities of the tube holder designed for receiving the tubes can be set at an interval which is compatible with an automated system for manipulating and moving the tubes.

The supporting tray can be made of any rigid material capable of supporting the tube receiver. The supporting tray can be filled with media designed to optimize plant growth in a bioreactor. Alternatively, the media can be placed directly in the rooting tube, such that the supporting tray is empty.

Medium and methods used for plant micropropagation have been described at least in M, R. Ahuja, Micropropagation of woody plants, Springer, 1993, ISBN 0792318072, 9780792318071; Narayanaswamy, Plant cell and tissue culture, Tata McGraw-Hill Education, 1994, ISBN 0074602772, 9780074602775; Singh and Kumar, Plant Tissue Culture, APH Publishing, 2009, ISBN 8131304396, 9788131304396; Y. P. S. Bajaj, High-tech and micropropagation V, Springer, 1997, ISBN 3540616063, 9783540616061; Tngiano and Gray, Plant Tissue Culture, Development and Biotechnology, CRC Press, 2010, ISBN 1420083260, 9781420083262; Gupta and Ibaraki, Plant tissue culture engineering Volume 6 of Focus on biotechnology, Springer, 2006, ISBN 1402035942, 9781402035944; Jam and Ishii, Micropropagation of woody trees and fruits Volume 75 of Forestry sciences, Springer, 2003, ISBN 1402011350, 9781402011351; and Aitken-Christie et al., Automation and environmental control in plant tissue culture, Springer, 1995, ISBN 0792328418, 9780792328414, each of which is incorporated herein by reference in its entirety.

The physical state of the media can vary by the incorporation of one or more gelling agents. Any gelling agent known in the art that is suitable for use in plant tissue culture media can be used. Agar is most commonly used for this purpose. Examples of such agars include Agar Type A, E or M and Bacto™ Agar. Other exemplary gelling agents include carrageenan, gellan gum (commercially available as PhytaGel™, Gelrite® and Gelzan™), alginic acid and its salts, and agarose. Blends of these agents, such as two or more of agar, carrageenan, gellan gum, agarose and alginic acid or a salt thereof also can be used. In some embodiments, no gelling agent or very little gelling agent is used for a liquid medium.

Also disclosed herein is a an automated device for the propagation of plant material, the device comprising: a laser unit, wherein said laser unit comprises: power supply; stage controller; computer connected to said stage controller; and laser cutter; a first conveyor belt for receiving propagules which were cut by said laser cutter into a first bioreactor; a second conveyor belt for delivering propagules which are contained in a bioreactor, wherein said propagules are to be cut by said laser cutter.

The laser cutter can be used in conjunction with the bioreactor and rooting tubes disclosed herein.

### Example Methods of Operations

Disclosed herein is a general method for the propagation of plant material. Therefore, in a broad sense, disclosed herein is a method comprising obtaining plant material from a suitable source, depositing said material in a tube, and placing the tube in a TIB device as disclosed herein. The plant material can be placed within the tube before it is placed in the tube holder, or this may occur after the tube has been secured in the tube holder. The entire TIB can then be placed under suitable conditions for plant propagation. This process is referred to herein as “Process 1.”

The plant propagule can be allowed to incubate under these conditions for a specified period of time, which can include 1, 2, 3, 4, 5, 6, 7, 8, 9, 10, 11, 12, 13, 14, 15, 16, 17, 18, 19, 20, or more days. Once the plant has reached a suitable size, it can be further propagated, for example planted in a proper substate to continue to grow (e.g., “Process 2” 103), or it can be divided, and the divided parts can then be placed back in a TIB for further incubation (e.g., “Process 3” 104). This process of dividing the plant propagule and returning it to a TIB can be repeated 2, 3, 4, 5, 6, 7, 8, 9, 10, or more times, or indefinitely. The method disclosed herein can be used with a plant growing system, such as within the system shown in FIG. 1C, which provides conditions suitable for plant propagation. The device disclosed herein can be part of a non-automated system (e.g., TIB reservoir that is manually refilled) automated system (e.g., automated TIB refill).

Referring to FIG. 1A, “process 1” 102 may involve the positioning the propagule 106 into the tube 108 at the correct level inside the tube the tubes being fixed into the tube-holder 110 where the top of the tube-holder comprise cavities that fits the tubes at the diameter of the tube 114 to position the tube at the optimal vertical position relative to the holder. The tubes are arranged in a pattern that is compatible with the geometrical design of the tray receiving the rooted propagules ex vitro 116.

The holder with tubes is transferred to the TIB for rooting and shoot development, or for mature somatic embryo germination. Examples of how somatic embryos can be generated in a TIB can be found in PCT Publication No. WO2011042888A2, herein incorporated by reference in its entirety.

Therefore, the method disclosed herein encompasses a method of placing a plant propagule in a tube, wherein said tube is comprised of a biodegradable material; and placing the tube in a vertical manner into a tube holder, wherein said tube holder comprises a bottom layer comprising a supporting tray, and a top layer comprising a tube receiver, wherein said tube receiver comprises cavities sized and configured for receiving tubes, and wherein the supporting tray comprises growth media, wherein the distal end of the tube is in contact with the germination media; and placing the tube holder containing the tube with the plant propagule under one or more conditions which are favorable for plant propagation, thereby propagating the plant.

Referring to the schematic device shown in images 150, 152, 154, “process 2” 103 may commence when the process of forming a viable root and/or shoot is complete, and the target for the process is planting of the propagule. The tube holder is removed from the TIB and the holder associated with the substrate tray to exactly position the tubes holding rooted propagules into the substrate. The tube holder is removed when the tubes have been positioned into the planting substrate. Again, this can be automated or can be done by hand. An image of the device used to accomplish this can be seen in FIG. 1C. Images 150, 152, 154 respectively illustrates different varieties of blueberry have been multiplied in vitro in V-Germination bioreactors (provided by Bioautomaton Systems Inc.). A. Shoot growth and root development in the TIB. B. Shoot growth on solid medium. C. Plants derived from TIBs established at nursery.

FIGS. 4A-C show a V(vertical)-germination bioreactor in several views. This research model holds 30 tubes for germination of somatic embryos, shoot multiplication or rooting of cuttings.

FIG. 5 is an image of a V-germination bioreactor showing root growth of germinated Norway spruce somatic embryos inside planting-tubes and shoot development.

FIG. 6 shows the germination plant box. The V-germination bioreactor shell is shown without connections with test propagules (Araucaria cuttings) for use with the automated Germination Platform. This plant box can hold 1080 tubes for germination of somatic embryos, shoot development or rooting of cuttings. FIG. 6 shows the entire box and a blown-up image showing the individual tubes with plants in them.

FIG. 1C is an image showing the setup of the Germination Platform in culture room.

“Process 3” 104 may commence when the process of forming enough shoot for shoot cuttings is complete and the target for the process is generating cuttings. The propagule inside the holder has formed a sufficiently developed shoot or roots that such can be cut to generate new shoots/roots. The cutting device is described is described in relation to FIGS. 2, 3A, and 3B. The cuttings generated in “Process 3” 104 may be placed in the holder according to “Process 1” 102.

Further disclosed is a method for automated laser cutting of a plant propagule, the method comprising: exposing a plant propagule contained in a bioreactor to a laser cutter, wherein said bioreactor comprises: a tube holder, wherein said tube holder comprises a bottom layer comprising a supporting tray, and a top layer comprising a tube receiver, wherein said tube receiver comprises cavities sized and configured for receiving tubes; two or more tubes, wherein said tubes are formed of biodegradable material, and further wherein the tubes are removably engaged by the cavities of the tube receiver, wherein a proximal end of the tube is open for receiving a plant propagule, and the distal end of the tube resides in the supporting tray of the tube holder.

There are many ways in which the propagule can be cut. For example, the propagule for cutting can be removed from the tube by an automated process and cut. In another example, the propagule can remain in the biodegradable tube, and the entire tube can be cut. The newly divided propagules can both be reinserted into a tube, or can be placed in a different substrate. Alternatively, either one of the divided propagules can be propagated further while the other portion is discarded or otherwise disposed of. The mean size of the cutting can be about 0.5 mm, about 1 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10.5 mm, 11 mm, 11.5 mm 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm and 20.0 mm.

### Examples Rooting Micro Cuttings

An example for using the method and device is given for rooting micro cuttings of blueberry highbush (V. cortymbosum L. hybrids) and Vaccinium sp. Micro cuttings at 20-25 mm length (FIG. 1) were harvested from in vitro cultures of blueberry and placed into tubes in a tube holder in a temporary immersion bioreactor. Micro cuttings were cultivated with the basal standard culture medium for shoot multiplication suitable for the specific cultivars. Micro cuttings were cultivated with the same basal culture medium but with any required supplements for root induction and growth suitable for the specific cultivar. The culture medium in the temporary immersion bioreactors is liquid and therefore the shoot elongation and subsequent root development is supported by liquid culture medium.

Elongation occurred at a higher rate and with more and larger leaves in the temporary immersion bioreactors than in the control cultures on solidified culture medium of the same composition shown in FIGS. 2A and B. Root induction and growth was earlier, more vigorous and occurred at a higher frequency in the temporary immersion bioreactors than in the control cultures on solidified culture medium of the same composition. Micro cuttings with developed root systems can be successfully transferred to ex vitro conditions and planted in compost. Growing plants were established and continued to grow as shown in FIG. 2C. The rooted micro cuttings from control cultures were overall smaller and showed less growth after planting.

Examples of using the method and device is also given for Stevia. Stevia plants were established directly in vitro from seeds. Cuttings from the in vitro plants were placed directly in the tubes for shoot growth and rooting. Shoot growth was much faster in the TIB and roots started forming in one week. Controls on solid medium showed slower shoot and root growth.

Examples of using the method and device is also given for somatic embryo germination. Somatic embryos of Norway spruce (Picea abies) and larch (Larix sp.) gemrinated well in the TIBs and formed substantial and straight roots. Root formation during germination on solid medium was slower and usually not straight.

### Proof-of-Concept System

A proof-of-concept device for automated cutting of bioreactor-grown plants has been fabricated. As part of this prototype, a CO2 laser for cutting the in vitro plants without contamination of any type is being used. The different components are outlined in the schematic. This device can also be used to cut the root of germinants from somatic embryos. Normally the trays are lined up in two rows, where one row will have trays are full.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth 10 reference in the list. All references cited and discussed in this specification are incorporated herein by reference in their entireties and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Further background description of various processes described herein are provided in the following references, each of which are incorporated by reference herein in its entirety.

### REFERENCES

Businge, E., Trifonova, A., Schneider, C., Rödel, P., Egertsdotter, U. 2017. Evaluation of a New Temporary Immersion Bioreactor System for Micropropagation of Cultivars of Eucalyptus, Birch and Fir. Forests 2017, 8(6), 196; doi:10.3390/f8060196.
Debnath, S. C. 2017. Temporary immersion and stationary bioreactors for mass propagation of true-to-type highbush, half-high, and hybrid blueberries (Vaccinium spp.), The Journal of Horticultural Science and Biotechnology, 92:1, 72-80, DOI:10.1080/14620316.2016.1224606.
Mamun, N. H. A., Aidun, C. K., Egertsdotter, U. 2018. Improved and synchronized maturation of Norway spruce somatic embryos in temporary immersion bioreactors. In vitro Cell Dev Biol. issue 1 suppl. doi:org/10.1007/s11627-018-9911-4
PCT Patent application no. PCT/IB2015/059811, published as WO/2016/098083.

1. A device comprising a temporary immersion bioreactor, wherein said bioreactor comprises:
a. a tube holder, wherein said tube holder comprises a bottom layer comprising a supporting tray, and a top layer comprising a tube receiver, wherein said tube receiver comprises cavities sized and configured for receiving tubes;
b. two or more tubes, wherein said tubes are formed of biodegradable material, and further wherein the tubes are removably engaged by the cavities of the tube receiver, wherein a proximal end of the tube is open for receiving a plant propagule, and the distal end of the tube resides in the supporting tray of the tube holder.

2. The device of claim 1, wherein the tube is designed of material that supports stimulation of root formation.

3. The device of claim 1, wherein the propagule is a shoot or root cutting.

4. The device of claim 1, wherein the propagule is a somatic embryo.

5. The device of claim 1, wherein the propagule is manipulatable inside the tube via the proximal end.

6. The device of claim 1, wherein the tube receiver is comprised of flexible material.

7. The device of claim 5, wherein the flexible material is mesh.

8. The device of claim 6, wherein the cavities hold the tubes directly.

9. The device of claim 1, wherein germination media is in the supporting tray.

10. The device of claim 1, wherein germination media is in the tubes.

11. The device of claim 9, wherein said germination media is liquid or gel.

12. (canceled)

13. The device of claim 9, wherein the distal end of the tube has one or more openings which allows for germination media from the supporting media to enter the tube.

14. The device of claim 13, wherein the one or more openings is sized to receive a root of the plant propagule.

15. A method of propagating plants, the method comprising:
a. placing a plant propagule in a tube, wherein said tube is comprised of a biodegradable material;
b. placing the tube in a vertical manner into a tube holder, wherein said tube holder comprises a bottom layer comprising a supporting tray, and a top layer comprising a tube receiver, wherein said tube receiver comprises cavities sized and configured for receiving tubes, and wherein the supporting tray comprises growth media, wherein the distal end of the tube is in contact with the germination media; and
c. placing the tube holder containing the tube with the plant propagule under one or more conditions which are favorable for plant propagation, thereby propagating the plant.

16. The method of claim 15, wherein the tube is designed of material that supports stimulation of root formation.

17. The method of claim 15, wherein the propagule is a shoot or root cutting.

18. The method of claim 15, wherein the propagule is a somatic embryo.

19. The method of claim 15, wherein the propagule is manipulatable inside the tube via the proximal end.

20. The method of claim 15, wherein the tube receiver is comprised of flexible material.

21-30. (canceled)

31. A method of propagating plants, the method comprising, after step c) of claim 1, placing the plants in an appropriate growth media.

32-69. (canceled)
